(19) United States
(12) Patent Application Publication
(10) Pub. No.: US 2010/0113572 A1
(43) Pub. Date: May 6, 2010

(54) PHOTOREACTIVE RU (II) COMPLEXES ANCHORED ON OLIGONUCLEOTIDES, METHOD FOR OBTAINING THEM AND USE THEREOF

(76) Inventors: Eric Defrancq, Saint Pierre d'Allevard (FR); Cécile Moucheron, Bruxelles (BE); Stéphane Rickling, Hirtzfelden (FR); Andrée De Mesmaeker(Kirsch), Bruxelles (BE)

Correspondence Address: MERCHANT & GOULD PC, P.O. BOX 2903, MINNEAPOLIS, MN, 55402-0903, US

(21) Appl. No.: 12/531,571

(22) PCT Filed: Mar. 5, 2008
(86) PCT No.: PCT/EP08/52667

§ 371 Date: Sep. 16, 2009

(30) Foreign Application Priority Data
Mar. 16, 2007 (EP) 07104297.2

(51) Int. Cl. A61K 31/7088; C07H 21/00; A61K 31/7135; A61P 35/00

(52) U.S. Cl. 514/44 R; 536/23.1; 536/24.5

(57) ABSTRACT

Photoreactive Ru (II) complexes are anchored on a G-containing oligonucleotide, e.g. compound 1. The method for obtaining them and their use, includes targeting specific nucleotide sequences that become photocrosslinked to the photoreactive Ru (II) complex anchored to the G-containing oligonucleotide.

### FIELD OF THE INVENTION

The present invention is related to a compound made of a photoreactive Ru (II) complex bound (anchored) to G-containing oligonucleotides, the method for obtaining them and their use, especially for targeting specific nucleotide sequences involved in hyperproliferative disorder and to avoid their expression in prokariote or eukaryote cells (especially in bacterial cells, plant cells, animal cells, fungi cells, including yeast cells).

A preferred use of these oligonucleotides is in the field of anti-tumoural therapy and possibly prevention.

### BACKGROUND OF THE INVENTION

Ru(II) complexes containing at least two Tap (1,4,5,8-tetraazaphenanthrene) ligands are known to be oxidant enough in their excited state to photo-oxidize a guanine unit of DNA molecule (oligonucleotide); such a ruthenium complex anchored on one strand of a hybridized oligonucleotide, without guanine, was able to react under illumination with a guanine situated on the complementary strand, which leads to a photocrosslinking of both strands that may be used in anticancer therapy (antisense strategy).

### STATE OF THE ART

WO 2004/037907 describes luminescent metal ion complexes and components thereof (including oligonucleotides tethered to these complexes) which are useful in detection and isolation of target analytes, such as biomolecules.

The European Patent EP 0 733 058 describes the selective modification of nucleic acids at specific sites with redox active moieties, such as transition metal complexes. This European Patent describes an electron donor and/or an electron acceptor moiety that are covalently bound, preferably along the ribose-phosphate backbone of the nucleic acids sequence at predetermined positions. These complexes possess unique structural features which enable the use of an entirely new class of bioconductors and diagnostic probes.

The publication of Lentzen & al, (Journal of Biological Inorganic Chemistry Volume 9 p. 100-108 (2004)) describes the determination of DNA guanine sites forming photo-adducts with Ru(II)-labeled oligonucleotides. In this publication, the inventors describe oligonucleotides containing photo-reactive Ru(II)complexes for use in antisense strategy. The synthesised oligonucleotides do not comprise any guanine in it, because these Ru(II) complexes are known to be reactive enough in the excited state to photo-oxidize the guanine unit present in the complementary DNA sequence.

The publication of Garcia-Fresnadillo & al (Bio-physical Journal Volume 82 p. 978-987 (2002)) describes luminescence quenching of Ru-Labeled oligonucleotides by targeted complementary strands. The probe sequences do not comprise any guanine units in order to avoid a photo-oxidation of one of the guanine unit present in the oligonucleotides probe-sequences.

### AIMS OF THE INVENTION

The present invention aims to provide compounds comprising oligonucleotides which are able to lead to photocrosslinking with complementary nucleotide sequences to be targeted and inactivated, and which do not present the drawbacks of the state of the art.

A preferred aim of the present invention is to obtain compounds made of oligonucleotides which present a high specificity and are photo-inactivated, if no binding (hybridisation) with their corresponding specific nucleotide sequences to be targeted has been obtained.

A further aim of the present invention is to propose such compound which will reduce the possible side effects of oligonucleotides which are not bound (hybridized) to their corresponding nucleotide sequences, for instance by avoiding any possible binding (hybridization) upon anti-oncogenic sequences, if no binding has been obtained upon targeted oncogenic sequences.

### SUMMARY OF THE INVENTION

The present invention is related to a compound made of an oligonucleotide bound to (linked to or anchored on) a photoreactive Ru (II) complex Ru (L₁) (L₂) (L₃)²⁺

wherein L₁ and L₂ both comprise two pyrazinic moieties coordinated to the Ru(II) center according to formula I

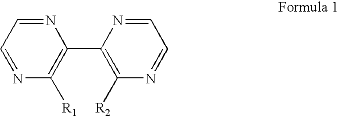

Wherein R1 and R2 are H or form between them a cycle having the formula II, formula III or formula IV

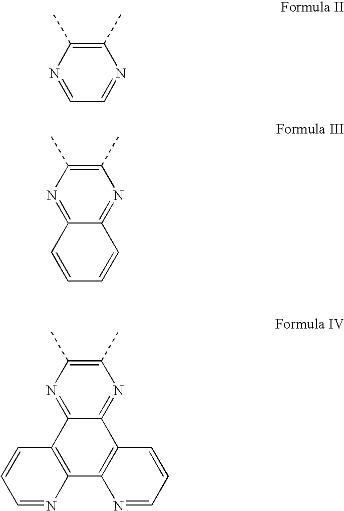

and wherein L₃ is any a di-imine polyazaaromatic ligand
and wherein these complexes are anchored via L₃ to oligonucleotides (defined also hereafter as Oligo), wherein these oligonucleotides are guanine-containing oligonucleotides, which means that these oligonucleotides comprise one or more guanine unit(s) (G base).

According to a preferred embodiment, in the compound of the invention, the photoreactive Ru (II) complex presenting the formula Ru (L₁) (L₂) (L₃)²⁺ comprises at least two ligands selected from the group consisting of the 1,4,5,8,9,12-hexaazatriphenylene (dipyrazino (1,3-f) (2′,3′-h)quinoxaline, the 2,2′ bipyrazine, the 1,10 phenanthrolino-[5,6-b]-1,4,5,8,9,12-hexaazatriphenylene or the 1,4,5,8-tetraazaphenanthrene (or pyrazino [2,3-f]-quinoxaline).

The oligonucleotide sequence of the compound is an antisense or RNAi oligonucleotide sequence (for gene silencing) and made of at least 8, 9, 10, 11, 12, 13, 14, 15, 20, 25, 30, 35, but less than 200, 150, 100, 50, 40 or 35 bases bound (linked or anchored) to the photoreactive Ru (II) complex on the 5′ or 3′ end (possibly through a linker sequence).

The present invention concerns also the method for obtaining these compounds wherein the binding (linkage or anchorage) between the photoreactive complex and the oligonucleotides is obtained with a chemical linker having a suitable end for allowing the binding to this complex.

The present invention is also related to a pharmaceutical composition or a diagnostic kit (kit of parts or apparatus) comprising the compound (II) above described and possibly a photon (hν) or a device that emits photons. The pharmaceutical composition may also comprise an adequate pharmaceutical carrier or diluent. In the diagnostic kit (or apparatus) according to the invention, one may also include a device that emits photons.

Another aspect of the present invention is related to the compound of the invention combined with a photon (that forms a reactive agent) for use as a medicament.

A further aspect of the present invention is related to the use of the pharmaceutical composition for the manufacture of a medicament in the treatment of a disease induced by expression of specific nucleotide sequences, such as an hyperproliferative disorder (i.e. cancer) induced by oncogenes, whose expression should be modulated, preferably decreased or suppressed and that are complementary to the sequences of the oligonucleotides present in the compound of the invention.

The adequate pharmaceutical carrier of the composition according to the invention or diluent is selected by the person skilled in the art according to the type of administration required upon specific cells or tissues.

The ratio between the amount of the adequate pharmaceutical carrier or diluent and the active compound is defined by the person skilled in the art, according to the administrated doses to be applied to the mammal subject, (including a human patient), the surface of the tumoural epithelium or the volume of the tumour to be treated and the possible side effects of the active compound upon the mammal subject, including a human patient.

Preferably, this adequate pharmaceutical carrier is present in a solid or liquid form and these oligonucleotides are possibly incorporated into suitable vectors (cationic vesicles, plasmid, virus, etc) with adjuvant(s) to transform (transfect) the cells.

Preferably, this pathology is an hyper-proliferative disorder or pathology (cancer) present in specific cells (tumour cells).

This hyper-proliferative disorder or pathology (cancer) treated by the composition of the present invention is a hyper-proliferative disorder or pathology (cancer), preferably affecting epithelial cells such as epithelial tumoural cells of the skin, of the digestive track (in particular tumours of rectum, colon, intestine, stomach, oesophagus, pharynx, or buccal cavity), tumoural cells of the lung, or tumoural cells affecting a genital or an urinary organ, in particular, the ureter, the urethra, the prostate, the urinary bladder or the uterus neck) or any other tissue that can be treated by the compound of the invention in addition to a photon, or by the device that may enter into a conduct of these organs affected by this tumour. Other examples of cells are prokaryote or eukaryotes cells, preferably pathogenic cells to form a reactive agent presenting nucleotides sequences to be targeted for a diagnostic or therapeutic purpose.

The treatment according to the invention comprises the steps of putting into contact the compound according to the invention with the cells to be treated and submitting these cells to an adequate light irradiation (photon emission). Preferably, this light irradiation is obtained by any suitable device that emits photons upon the area of the epithelium to be treated.

Preferably, this device emits light in the visible range for a sufficient time for obtaining an efficient treatment of the tumoural cell (through activation of the photoreactive complexes).

Advantageously, the compound and this device could be present in a kit of parts or an apparatus, possibly comprising an endoscope or catheter or any medical device which could be used for obtaining a contact between an epithelium comprising said tumoural cell and the (actived) compound elements of the pharmaceutical composition according to the invention under light irradiation (photon emission).

The present invention will be described in more details in the following non-limited examples, in reference to the enclosed figures.

### SHORT DESCRIPTION OF THE FIGURES

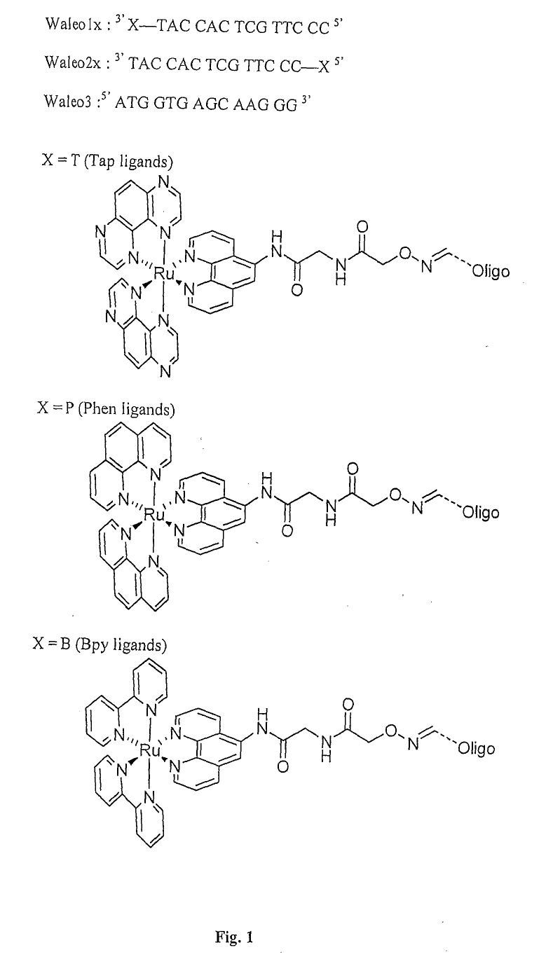

The FIGS. 1 to 3 show different compounds (Ru (II) oligonucleotides) according to the invention.

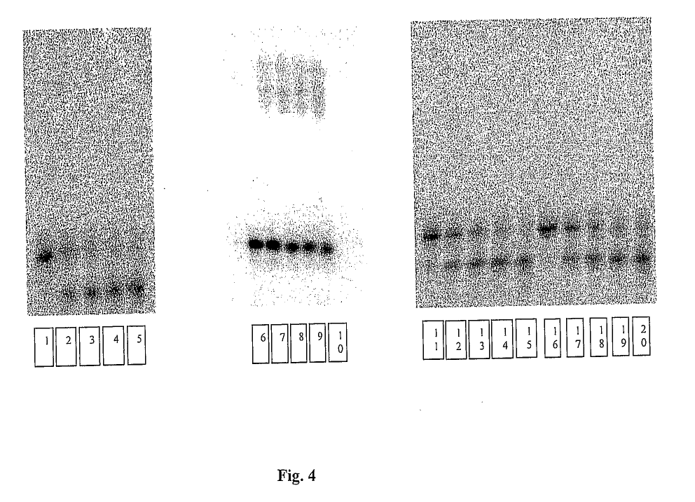

The FIG. 4 represents a polyacrylamide gel electrophoresis of the different oligonucleotides according to the invention. Denaturating PAGE experiments after 0, 5, 10, 15 and 30 min of illumination:
line 1 to 5: W1T* ss
line 6 to 10: W1T* hybridized with W3 (W1T ds)
line 11 to 15: W1T* and SC1
line 16 to 20: W1T* and SC2

### DETAILED DESCRIPTION OF THE INVENTION

A compound (photoactive Ru(II) complex containing at least two TAP (1,4,5,8-tetraazaphenanthrene) ligands anchored on an oligonucleotide containing a guanine unit) is used for the specific recognition of an oligonucleotide sequence and the formation, under visible light irradiation, of a photocrosslinking between this Ru-oligonucleotide and its complementary strand. The presence of a guanine base in the ruthenium-labeled oligonucleotide sequence implies an intramolecular photoreaction of this Ru-ODN (ODN=oligodéoxyribonucleotide) in the absence of the target sequence, even if it is in presence of any other G containing ODN. This work is the basis of a new type of intelligent drugs called “seppuku molecules”, which autodestruct if they do not manage to find their target, leading to a totally non-reactive and non-toxic species.

Three different Ru(II) complexes anchored on a 14-mer oligonucleotide, either in position 3′ or 5′ were obtained. The complexes used are Ru(Tap)₂Phen²⁺ (which is photoreactive with the guanine) and Ru(Bpy)₂Phen²⁺ and Ru(Phen)₂Phen²⁺ (wick are not photooxydant enough to react with a G base, under illumination) [Bpy=2,2′ bipyridine, Phen=1,10-phenanthroline, Phen″=5-((N-(ter-butoxycarbonyl)-O-(carboxymethyl)hydroxylamine)glycinamido) -1,10-phenanthroline]. Ru(Tap)₂Phen²⁺ and Ru(Phen)₂Phen²⁺ are chemically bound to the 3′ or 5′ end of the ODN via an oxime bond. Ru(Bpy)₂Phen²⁺ is obtained by reacting Ru(Bpy)₂Cl₂ with the derivatized ligand Phen″ and is anchored to the oligonucleotides derivatized with an aldehyde function.

The single stranded and duplex solutions used for the spectroscopic studies were prepared at a concentration of 1.10⁻⁵ M in an aqueous buffer (Tris-HCl 10 mM, NaCl 150 mM, pH=7). All the measurements have been realised in 600 μL quartz cells (UV select, 1.0×0.2 cm). Illuminations have been performed with a Thermo Oriel Xe lamp (500 W) (Fairlight, The Netherlands) with a H₂O and aqueous KNO₂ filters. Absorption and emission spectra have been performed on a Perkin-Elmer Lambda 40 UV-Vis spectrometer and a Shimadzu RF-5001PC spectrofluorimeter equipped with a Hamamatsu R928 red-sensitive photomultiplier tube respectively.

Radiolabelling in 5′ position of the ODNs have been realised by treating with T4 polynucleotide kinase and [β³²P] ATP at 37° C. for 30 min. Hybridization, when necessary, has been performed by incubating the labeled ODN with its complementary strand at 85° C. for 5 min and at room temperature for at least 6 hrs. Illuminations for the PAGE experiments have been performed with a He/Cd laser (442 nm) (Melles Griot).

Polyacrylamide gel electrophoresis have been performed through a denaturing (urea 7 M) 20% polyacrylamide (19:1 ratio of acrylamide to bisacrylamide) with TBE (90 mM Tris-borate, pH=8, 2 mM EDTA) gel. DNA fragments were visualised by autoradiography with a Storage Phosphor Screen (Amersham) film and were counted with a Phosphor-Imager Storm 860 instrument.

The FIG. 1 shows the different Ru-oligonucleotides that have been synthesized. These Ru-ODNs have been called Waleo1 if the anchoring of the complex was done on the 3′ end and Waleo2 if the anchoring was done on the 5′ end. Moreover, if the complex anchored contains Tap ligands, T to the former name was added, if it contains Bpy ligands, a B was added and if it contains Phen ligands, a P was added, so to obtain 6 different Ru-ODNs : Waleo1T, Waleo2T, Waleo1B, Waleo2B, Waleo1P and Waleo2P; Waleo3 is their complementary strand. The important fact to notice is that the oligonucleotide used to support the Ru(II) complex contains a guanine unit which precisely is the base that is oxidized by the photo-oxidizing Ru(II) complexes, if the target complementary strand is not found.

The solutions of the different Ru-ODNs were first illuminated in an aqueous buffer and one may observe the evolution of the absorption and emission spectra as a function of the illumination time. 1.10⁻⁵ M solutions of single stranded and double stranded Waleo1x and Waleo2x (x=T, B or P) were prepared. Illumination of 500 μL of each solution was undertaken and absorption and emission spectra were recorded after 5, 15, 30 and 60 min of illumination. Typical spectra of Waleo1Pss, Waleo1Pds, Waleo1Tss and Waleo1Tds can be seen on the figures.

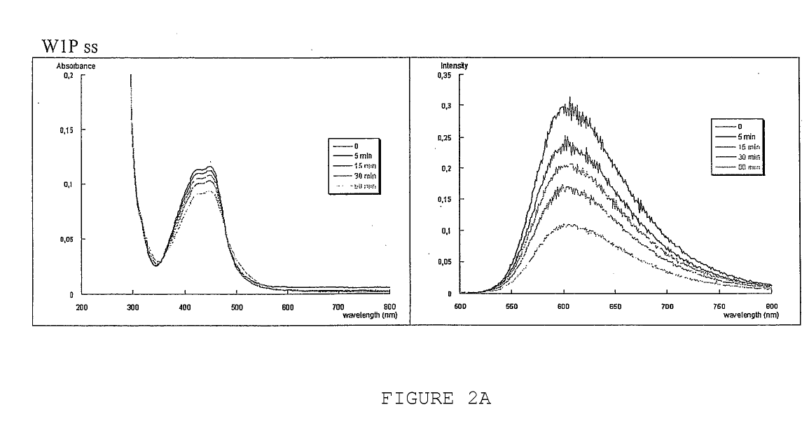

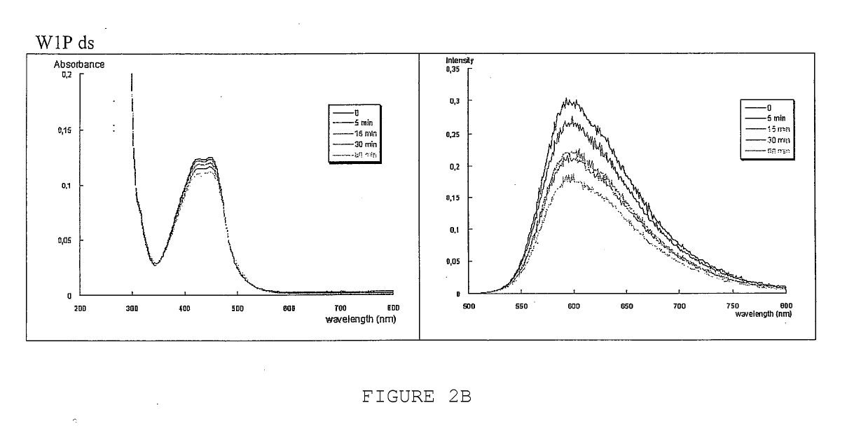

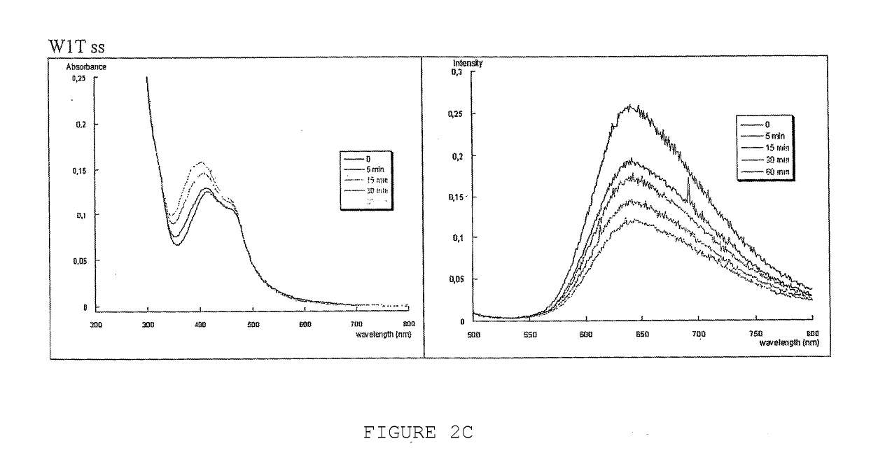

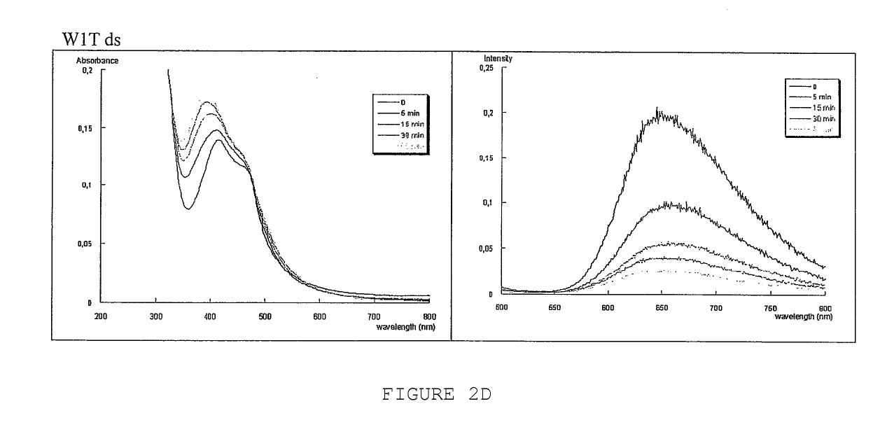

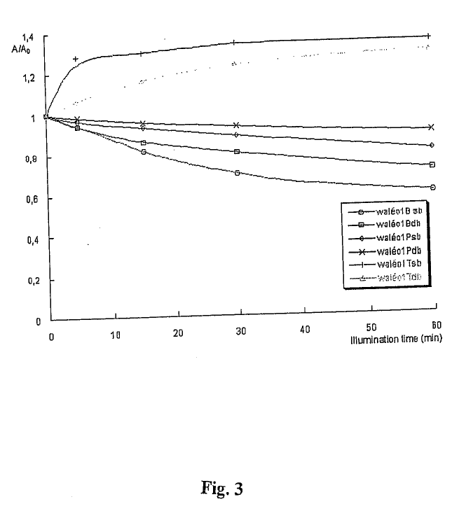

During illumination, the absorption spectrum of Waleo1P single stranded and Waleo1P double stranded (ds) undergoes a hypsochromic effect around 450 nm (MLCT absorption band) and a hyperchromic effect above 500 nm whereas the emission drops during the illumination, which is known to be characteristic of a photodechelation of the Ru(II) complex (FIG. 2). In contrast, the absorption spectra of Waleo1T ss shows a hyperchromic and hypsochromic effect of the MLCT absorption band around 420 nm. This is characteristic of the formation of a photoadduct; that means that the Ru(Tap)₂Phen²⁺ complex anchored on the ODN reacts, under illumination, with a G base. The origin of the photo-product can be double: either it is an intramolecular photo-product (the Ru(Tap)₂Phen²⁺ complex in its excited state reacts with the guanine of its own ODN strand) or it is an intermolecular photoadduct (the Ru(Tap)₂Phen²⁺ complex in its excited state reacts with the guanine of another Ru-ODN). The same observations are made from the absorption spectra of the double stranded Waleo1T sample under illumination with the only difference that the photoreaction with the single strand is faster than with the double strand (FIG. 3).

The results obtained with the 5′-modified ODNs are very similar to those described for the 3′-modified ODNs. The only difference is in the emission spectrum evolution of Waleo2Tds compared to Waleo1Tds. Waleo2Tds shows very poor emission compared to Waleo1Tds and its luminescence does not evolve during illumination. This may result from the direct vicinity of 3 Gs on the extremity of the 3′ end of the target strand. The presence of 3 guanines might favour the electron transfer between one of them and the excited ruthenium complex and thereby quenches the luminescence of its excited state.

To check the nature of the photoadduct obtained by illuminating Waleo1T alone and in presence of its complementary strand, polyacrylamide gel electrophoresis experiments were performed.

Therefore, 5′-³²P labeled Waleo1T and Waleo1Tds solutions were illuminated with a monochromatic laser (λ=452 nm) during 5, 10, 15 and 30 min (FIG. 4). Lane 1 is the native Waleo1T Ru-ODN. During illumination, Waleo1T is consumed and a new band appears migrating faster than the starting material (lanes 2 to 5). This observation is consistent with the hypothesis of intramolecular photoreaction of Waleo1T. After 30 min of illumination, 85% of intramolecular photoproduct were obtained.

When Waleo1T is hybridized with its complementary strand and illuminated as described, a new band appears on the electrophoresis gel, migrating much slower than native Waleo1T, consistent with a photocrosslinking product (lane 7 to 10). The amount of photocrosslinking is around 40% of the starting material after 30 min of illumination. On the other hand, no trace of the intramolecular photoproduct can be seen. The compounds Waleo1B, Waleo2B, Waleo1P and Waleo2P do not give rise to photocrosslinkings in the presence of Waleo3. Thus, Waleo1T under illumination undergoes an intramolecular reaction whereas the double strand leads selectively to the photocrosslinking product.

In a second step, the complementary strand (Waleo3) were replaced by another ODN strand containing one or more guanine bases in order to check the specificity of the photoactive molecule. Two scramble sequences SC1: 5′-TTT TCG TTT TAA ATT AT-3′ with 1 G and SC2: 5′-TAA ATT TAA GGA AAA AA-3′ with 2 Gs were used. After illumination, PAGE experiments showed absolutely no photocrosslinking product, but only intramolecular photoproduct could be detected (Lane 11 to 20).

In conclusion, Waleo1T photoreacts very specifically with its target sequence and leaves all other guanines containing oligonucleotides intact. In the presence of any other sequence than the targeted sequence, Waleo1T undergoes an intramolecular reaction rather than reacting with a non-recognized guanine. In other words, if the photo-active molecule does not find its target (its complementary strand), it kills itself and does not damage any other DNA sequence.

1.-10. (canceled)

11. A compound comprising oligonucleotide bound to a photoreactive Ru (II) complex Ru(L₁)(L₂)(L₃)²⁺ comprising at least two ligands according to formula I 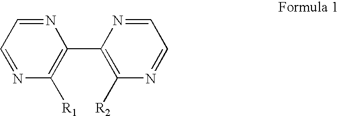
Wherein R1 and R2 are H or form between them a C═C double bound having the formula II or a cycle having the formula III, formula IV or formula V. 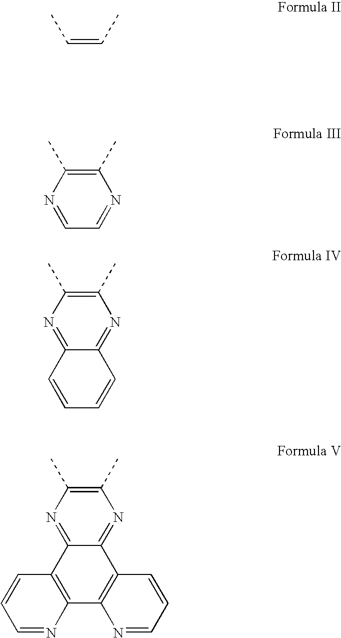
wherein L₃ is any di-imine polyazaaromatic ligand and wherein these complexes are anchored via L₃ to guanine-containing oligonucloetides.

12. The compound according to claim 11, wherein the photoreactive Ru (II) complex is anchored upon the oligonucleotide sequence by a chemical linker.

13. The compound according to any of the claim 11, wherein the oligonucleotide is an antisense oligonucleotide.

14. A diagnostic kit or apparatus comprising the compound according to claim 11.

15. The diagnostic kit or apparatus of claim 14 which further comprising a device that emits photons.

16. A pharmaceutical composition comprising an adequate pharmaceutical carrier or diluent, the compound according to claim 11 and a photon.

17. A method for the treatment and/or the prevention of an hyper-proliferative disorder which comprises administering the pharmaceutical composition of claim 16 to a mammal subject.

18. The method according to claim 17, wherein the hyper-proliferative disorder is a disorder affecting an epithelial cell.

19. The method according to claim 18, wherein the epithelial cell is a tumoural cell selected from the group consisting of a skin tumoural cell, a digestive track tumoural cell, lung tumoural cell and tumoural cell of the reproductive and/or urinary organ of a mammal.

20. The method according to the claim 17, wherein the mammal is a human.
